(19) United States
(12) Patent Application Publication
(10) Pub. No.: US 2019/0359582 A1
(43) Pub. Date: Nov. 28, 2019

(54) PROCESS FOR PRODUCING A TETRAHYDROFURAN COMPOUND COMPRISING AT LEAST TWO AMINE FUNCTIONAL GROUPS

(71) Applicant: RHODIA OPERATIONS, Paris (FR)

(72) Inventors: Peng LI, Shanghai (CN); Armin T. LIEBENS, Nancy (FR)

(21) Appl. No.: 16/472,365

(22) PCT Filed: Dec. 15, 2017
(86) PCT No.: PCT/CN2017/116465

§ 371 Date: Jun. 21, 2019

(30) Foreign Application Priority Data
Dec. 22, 2016 (CN) PCT/CN2016/111428

(51) Int. Cl. C07D 307/14; B01J 23/44; B01J 21/18; B01J 21/04; B01J 25/02; B01J 25/00

(52) U.S. Cl. CPC C07D 307/14; B01J 23/44; B01J 25/00; B01J 21/04; B01J 25/02; B01J 21/18

(57) ABSTRACT

The present invention concerns a process for preparing a tetrahydrofuran compound comprising at least two amine functional groups by reacting a furan compound comprising at least two nitrogen-containing functional groups with hydrogen in the presence of a hydrogenation catalyst.

### CROSS-REFERENCE TO RELATED APPLICATION

The present application claims priority to International Application No. PCT/CN2016/111428 filed on 22 Dec. 2016, the whole content of this application being incorporated herein by reference.

### TECHNICAL FIELD

The present invention concerns a process for preparing a tetrahydrofuran compound comprising at least two amine functional groups by reacting a furan compound comprising at least two nitrogen-containing functional groups with hydrogen in the presence of a hydrogenation catalyst.

### BACKGROUND

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of common general knowledge in the field.

2,5-Bis(aminomethyl)tetrahydrofuran is useful as a corrosion inhibitor. For example, it may be added to boilers or radiators to reduce the usual corrosive action which occurs therein. This diamine is also useful as an absorbent for acidic gases, such as CO₂, SO₂ and H₂S. Passage of a gas mixture containing such an acidic gas, through a column or bed of diamine will result in selective removal of the acidic gas. Nowadays, it is expected to be useful for making a variety of biobased polymers, see e.g., WO2013/007585.

2,5-Bis(aminomethyl)tetrahydrofuran could be obtained from different starting materials. Naemura et al. Chemistry Lett., 1985, 615-616 reports a three-step process starting from tetrahydrofuran-dicarboxylic acid to produce 2,5-bis(aminomethyl)tetrahydrofuran. Kohn et.al J. Org. Chem., 2002, 67, 1692-1695 discloses a new process with commercial starting material hexa-1,5-diene. U.S. Pat. Nos. 2,857,397 and 553,246 teaches this product could be made from cis-2,5-(hydroxymethyl)-tetrahydrofuran ditosylate. However, the reported prior arts are not ideal for commercialization production since the starting compounds above mentioned are not easy to obtain or processes are quite complicated.

Hydroxymethylfurfural (hereinafter referred as to HMF) is an attractive biomass feedstock for chemicals. WO2013/007585 reports 2,5-bis(aminomethyl)tetrahydrofuran could be synthesized starting from HMF. Disadvantageously, HMF is transferred into tetrahydrofuran-2,5-dimethanol in the presence of Raney nickel first. Then, additional steps are needed to prepare the final product, 2,5-bis(aminomethyl)tetrahydrofuran.

WO2015/175528 discloses bis(aminomethyl)tetrahydrofuran can be prepared by furan compounds comprising amine, imine or azide functional group in presence of Raney nickel. But the reaction has to be performed under harsh reaction condition, in which high pressure is necessary.

### INVENTION

It is therefore an objective of this invention to provide a process for producing a tetrahydrofuran compound comprising at least two amine functional groups, notably 2,5-bis(aminomethyl)tetrahydrofuran with desired characteristics such as mild reaction condition, high conversion and selectivity and overcome the drawbacks in prior arts.

The present invention concerns a one-step process for preparing a tetrahydrofuran compound comprising at least two amine functional groups by reacting a furan compound comprising at least two nitrogen-containing functional groups with hydrogen in the presence of a hydrogenation catalyst, wherein the hydrogenation catalyst comprises:
at least one noble metal element in elemental form and/or at least one noble metal compound of at least one noble metal element, or
at least one metal element in elemental form and/or at least one metal compound of at least one metal element and a dopant, wherein the metal element is chosen in the group consisting of (i) elements of group IA, IIA, IIIA, IVA, VA, VIA and VIIA of the Periodic Table, (ii) elements of groups IB, IIB, IIIB, IVB, VB, VIB, VIIB and VIIIB of the Periodic Table, (iii) lanthanides, (iv) actinides, and (v) any combination thereof.

Without wishing to be bound by any theory, the reaction condition is milder than that is described in any prior art. Specifically, high hydrogen gas pressure is not necessary in invented process. At the same time, the conversion and selectivity is comparatively high and thus is more suitable for commercialization production compared to any prior arts reported.

Other characteristics, details and advantages of the invention will emerge even more fully upon reading the description which follows.

### DEFINITIONS

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The term “and/or” includes the meanings “and”, “or” and also all the other possible combinations of the elements connected to this term.

Throughout the description, including the claims, the term “comprising one” should be understood as being synonymous with the term “comprising at least one”, unless otherwise specified, and “between” should be understood as being inclusive of the limits.

As used herein, the term “dopant” refers to doping agent added to catalyst materials in small amounts to improve their activity, selectivity and/or stability.

As used herein, metals of group IB, IIB, IIIB, IVB, VB, VIB, VIIB and VIIIB are often referred to as transition metals. This group comprises the elements with atomic number 21 to 30 (Sc to Zn), 39 to 48 (Y to Cd), 72 to 80 (Hf to Hg) and 104 to 112 (Rf to Cn).

As used herein, the lanthanides encompass the metals with atomic number 57 to 71 and the actinides encompass the metals with the atomic number 89 to 103.

As used herein, furan compound is defined as a compound comprising at least a furan group.

As used herein, tetrahydrofuran compound is defined as a compound comprising at least a tetrahydrofuran group.

Should the disclosure of any patents, patent applications, and publications which are incorporated herein by reference conflict with the description of the present application to the extent that it may render a term unclear, the present description shall take precedence.

### DETAILS OF THE INVENTION

The nitrogen-containing functional group of present invention is not particularly limited. It could notably be chosen in the group consisting of amine, imine, azide, hydrazone, nitro and oxime. Preferably, the nitrogen-containing functional group is chosen from the group consisting of amine, azide, and oxime and more preferably oxime.

Examples of furan compound comprising at least two nitrogen-containing functional groups according to invention are 2,5-bis(azidomethyl)furan, furan-2,5-dicarbaldehyde dioxime, 2,5-bis(hydrazonomethyl)furan, 2,5-bis(aminomethyl)furan, 3-((5-(aminomethyl)furan-2-yl)methoxy)propan-1-amine, 3,3′-((furan-2,5-diylbis(methylene))bis(oxy))bis(propan-1-amine), 3,3′-((furan-2,5-diylbis(methylene))bis(oxy))dipropanenitrile, ((oxybis(methylene))bis(furan-5,2-diyl))dimethanamine and 3,3′4((oxybis(methylene))bis(furan-5,2-diyl))bis(methylene))bis(oxy))dipropanenitrile.

Preferably, furan compound comprising at least two nitrogen-containing functional groups may be chosen in the group consisting of 2,5-bis(azidomethyl)furan, furan-2,5-dicarbaldehyde dioxime, 2,5-bis(hydrazonomethyl)furan and 2,5-bis(aminomethyl)furan and may be more preferably furan-2,5-dicarbaldehyde dioxime.

Examples of tetrahydrofuran compound comprising at least two amine functional groups according to invention are 2,5-bis(aminomethyl)tetrahydrofuran, 3-((5-(aminomethyl)tetrahydrofuran-2-yl)methoxy)propan-1-amine, 3,3′-(((tetrahydrofuran-2,5-diyl)bis(methylene))bis(oxy))bis(propan-1-amine), ((oxybis(methylene))bis(tetrahydrofuran-5,2-diyl))dimethanamine and 3,3′-((((oxybis(methylene))bis(tetrahydrofuran-5,2-diyl))bis(methylene))bis(oxy))bis(propan-1-amine).

Preferably, tetrahydrofuran compound comprising at least two amine functional groups may be 2,5-bis(aminomethyl)tetrahydrofuran.

It is to be understood that all stereoisomers of tetrahydrofuran compound comprising at least two amine functional groups can be obtained in the method according to the invention, either in admixture or in pure or substantially pure form. Furthermore, tetrahydrofuran compound comprising at least two amine functional groups encompasses both cis-isomers and trans-isomers.

As previously expressed, the hydrogenation catalyst of present invention could comprise at least one noble metal element in elemental form and/or at least one noble metal compound of at least one noble metal element.

In present invention, the noble metals are metals that are normally valuable and resistant to corrosion and oxidation in moist air. It could be chosen from the group consisting of ruthenium, rhodium, palladium, silver, osmium, iridium, platinum and gold.

In one embodiment, the hydrogenation catalyst of present invention may comprise one and only one noble metal element in elemental form. Preferably, the only one noble metal may be chosen in the group consisting of palladium, platinum, ruthenium and rhodium.

In another embodiment, the hydrogenation catalyst of present invention may comprise a mixture comprising at least two noble metal elements in elemental form.

In present invention, the hydrogenation catalyst comprising at least one noble metal compound of at least one noble metal element may notably be a metal complex. “Metal complex” is a substance consists of a central atom or ion, which is usually metallic and is called the coordination center, and a surrounding array of bound molecules or ions, that are in turn known as ligands or complexing agents.

The metal complex is not particularly limited. It should be understood by the people having ordinary skill in the art may use any metal complex as a hydrogenation catalyst mentioned in prior arts, such as ruthenium-based transition metal complexes catalyst as described in US2015057450.

The hydrogenation catalyst comprising at least one noble metal element in elemental form and/or at least one noble metal compound of at least one noble metal element could be prepared on a support. The support is not particularly limited. For example, the support might be chosen in the group consisting of silica, alumina, ceria, titania, zirconia, carbon and graphite powder.

The loading amount of noble metal on support may be comprised from 0.01% to 20% by weight with respect to the total weight of supported catalyst. Preferably, the loading amount of noble metal on support may be comprised from 1% to 15% by weight with respect to the total weight of supported catalyst.

Examples of supported catalyst are Pd/C, Pt/C, Rh/C, Ru/C, Au/C, Pd/CeO₂, Pd/Al₂O₃, Pt/Al₂O₃, Rh/Al₂O₃, Ru/Al₂O₃ and Au/Al₂O₃.

As previously expressed, the hydrogenation catalyst of present invention could comprise at least one metal element in elemental form and/or at least one metal compound of at least one metal element and a dopant, wherein the metal element is chosen in the group consisting of (i) elements of group IA, IIA, IIIA, IVA, VA, VIA and VIIA of the Periodic Table, (ii) elements of groups IB, IIB, IIIB, IVB, VB, VIB, VIIB and VIIIB of the Periodic Table, (iii) lanthanides, (iv) actinides, and (v) any combination thereof.

In present invention, hydrogen is not included in metal element chosen in Group IA of the Periodic Table. Carbon is not included in metal element chosen in Group IVA of the Periodic Table. Nitrogen and phosphorus are not included in metal element chosen in Group VA of the Periodic Table. Oxygen, sulfur and selenium are not included in metal element chosen in Group VIA of the Periodic Table.

Some of the elements encompassed by the description above and understood to be metals for the purpose of the present invention, are sometimes also referred to as metalloids. The term metalloid is generally designating an element which has properties between those of metals and non-metals. Typically, metalloids have a metallic appearance but are relatively brittle and have a moderate electrical conductivity. The six commonly recognized metalloids are boron, silicon, germanium, arsenic, antimony, and tellurium. Other elements also recognized as metalloids include aluminum, polonium, and astatine. On a standard periodic table all of these elements may be found in a diagonal region of the p-block, extending from boron at one end, to astatine at the other.

In one embodiment, the hydrogenation catalyst of present invention may comprise one and only one metal element in elemental form and a dopant. The only one metal element in elemental form may be notably chosen in the group consisting of elements of groups IB, IIB and VIIIB of the Periodic Table. Preferably, the only one metal element in elemental form may be chosen in the group consisting of Pt, Pd, Rh, Ru, Au, Ag, Ni, Co, Fe, Zn and Cu.

In another embodiment, the hydrogenation catalyst of present invention may comprise a mixture comprising at least two metal elements in elemental form and a dopant.

In still another embodiment, the hydrogenation catalysts of present invention may comprise a metal alloy comprising at least two metal elements in elemental form and a dopant.

A metal alloy can be viewed as a solid metal-solid metal mixture wherein a primary metal acts as solvent while other metal(s) act(s) as solute; in a metal alloy and wherein the concentration of the metal solute does not exceed the limit of solubility of the metal solvent.

Preferably, the two metal elements of hydrogenation catalyst comprising a mixture comprising at least two metal elements in elemental form or a metal alloy comprising at least two metal elements in elemental form and a dopant may be chosen in the group consisting of elements of groups IIIA, IVA, VA, VIA, IB, IIB, IIIB, IVB, VB, VIB, VIIB and VIIIB of the Periodic Table.

More preferably, the two metal elements of hydrogenation catalyst comprising a mixture comprising at least two metal elements in elemental form or a metal alloy comprising at least two metal elements in elemental form and a dopant may be chosen in the group consisting of elements of groups IB, IIB, IIIB, IVB, VB, VIB, VIIB and VIIIB of the Periodic Table and metalloids.

In a preferred embodiment, the two metal elements of hydrogenation catalyst comprises a mixture comprising at least two metal elements in elemental form or a metal alloy comprising at least two metal elements in elemental form and a dopant may be chosen in the group consisting of Pt, Pd, Rh, Ru, Au, Ag, Ni, Co, Fe, Cu, Sn, B, Al, Si, Sb, Bi and Ge.

The metal alloy may be notably selected from the group consisting of Pt—Au, Pt—Pd, Pt—Sn, Pt—Bi, Pt—Fe, Rh—Ag, Rh—Au, and Raney nickel alloys. Raney nickel alloys are preferable among these alloys.

Raney nickel is an alloy containing catalytically active nickel and a catalytically inactive component, such as aluminum or silicon. The Raney nickel alloy always has a very high surface area and also contains hydrogen gas (H₂) adsorbed on the nickel surface.

The Raney nickel alloy mentioned above may notably be Ni—Al, Ni—Si, Ni—Sn, Ni—Co—Si alloys. Among these, Ni—Al alloy is more preferable.

In a preferred embodiment of the invention, the catalyst may have a total aluminum content of at most 10% by weight with respect to the total weight of Ni—Al Raney nickel alloy.

In another preferred embodiment of the invention, the total aluminum content may be greater than or equal to 1% by weight with respect to the total weight of Ni—Al Raney nickel alloy.

Preferably, the total aluminum content may be comprised from 1% to 10% by weight with respect to the total weight of Ni—Al Raney nickel alloy. Even more advantageously, the total aluminum content may be comprised from 2% to 8% by weight with respect to the total weight of Ni—Al Raney nickel alloy.

According to any one of the invention embodiments, the metal alloy may preferably be a Raney cobalt alloy.

The Raney cobalt is an alloy containing catalytically active cobalt and a catalytically inactive component, such as aluminum or silicon.

The Raney cobalt alloy suitable for the present invention may notably be a Co—Al Raney cobalt alloy. In a preferred embodiment, the Co—Al Raney cobalt alloy may have a total aluminum content of at most 10% by weight with respect to the total weight of the Co—Al Raney cobalt alloy. In another preferred embodiment, the total aluminum content may be greater than or equal to 1% by weight with respect to the total weight of the Co—Al Raney nickel alloy.

Preferably, the total aluminum content may be comprised from 1% to 10% with respect to the total weight of the Co—Al Raney cobalt alloy. More preferably, the total aluminum content may be comprised from 2% to 8% by weight with respect to the total weight of the Co—Al Raney cobalt alloy.

In present invention, the dopant may notably be a metal element. Among the conventional dopants in this field, exemplary are elements of groups IVB, VB, VIB, VIIB and VIIIB of the Periodic Table. Preferably, the dopant may notably be chosen from the group consisting of Zn, Fe, Ti, Mo, V, Cr, Co, Mn and combinations thereof.

The doped catalyst notably refers to Ni—Al Raney nickel alloy comprising metallic dopant elements, iron and chromium. According to one preferred feature of the invention, the catalyst could be the one used as a reference catalyst in Example 1 of US 2011230681.

The doped catalyst notably refers to a Co—Al Raney cobalt alloy comprising metallic dopant elements, such as nickel, iron and chromium.

In some embodiments, the doped catalyst can be a Co—Al Raney cobalt alloy comprising dopant elements nickel and chromium. The total nickel content may be comprised from 1% to 4% by weight with respect to the total weight of the Co—Al Raney cobalt alloy. The total chromium content may be comprised from 1% to 4% by weight with respect to the total weight of the Co—Al Raney cobalt alloy.

The amount of hydrogenation catalyst comprising at least one noble metal element in elemental form and/or at least one noble metal compound of at least one noble metal element, expressed by the ratio of the weight of noble metal comprised in catalyst to the weight of furan compound comprising at least two nitrogen-containing functional groups may be comprised from 0.0001:1 to 0.1:1 and preferably from 0.0005:1 to 0.02:1.

The amount of hydrogenation catalyst comprising at least one metal element in elemental form and/or at least one metal compound of at least one metal element and a dopant, expressed by the ratio of the weight of metal comprised in catalyst to the weight of furan compound comprising at least two nitrogen-containing functional groups, can vary, for example, from 0.0001:1 to 2:1

In a particular embodiment, when doped Raney nickel alloy is employed in the invented process, the amount of catalyst, expressed by the ratio of the weight of metal comprised in catalyst to the weight of furan compound comprising at least two nitrogen-containing functional groups may be comprised from 0.05:1 to 1:1.

The hydrogen employed is more particularly pure hydrogen. By the expression “pure hydrogen” is intended a gas containing at least 99% hydrogen and more especially at least 99.9% hydrogen.

The reaction may preferably be conducted in a non-oxidizing atmosphere. As used herein “non-oxidizing atmosphere” means any atmosphere that excludes oxygen and does not lead to the formation of undesirable side reaction products. Suitable non-oxidizing atmospheres which can be provided are, for example, inert gases such as N₂, He, Ne or Ar.

According to present invention, the hydrogen has a gas pressure which is of at most 80 bars. It could be preferably comprised from 5 to 80 bars and more preferably from 10 and 60 bars.

In one embodiment, when hydrogenation catalyst comprising at least one noble metal element in elemental form and/or at least one noble metal compound of at least one noble metal element is employed in the invented process, the hydrogen gas pressure may be comprised from 20 to 60 bars.

In another embodiment, when hydrogenation catalyst comprising at least one metal element in elemental form and/or at least one metal compound of at least one metal element and a dopant employed is employed in the invented process, the hydrogen gas pressure may be comprised from 10 to 50 bars.

Solvent could be optionally used in present invention for dissolving furan compound comprising at least two nitrogen-containing functional groups. The solvent is typically chosen based on its ability to dissolve the reactants. It could be chosen in a group consisting of water, alcohols, ether, ester, ketone and any combination thereof.

According to present invention, the conversion of furan compound comprising at least two nitrogen-containing functional groups may reach at least 40%. The conversion could be preferably comprised from 40% to 100% and more preferably from 80% to 100%.

Conversion corresponds to the total amount of reactant that was converted during the reaction. Conversion may be determined for instance by the way of dividing the amount of reactant converted by the amount of reactant supplied.

According to present invention, the selectivity of tetrahydrofuran compound comprising at least two amine functional groups may be of at least 60%. The selectivity of tetrahydrofuran compound comprising at least two amine functional groups could be preferably comprised from 60% to 100% and more preferably from 70% to 90%.

Selectivity corresponds to transformation of the reactant to the desired product divided by the overall conversion of the reactant. Selectivity may be determined for instance by the way of dividing the number of moles of the desired product by the total amount of moles of all products obtained (desired and undesired products).

The reaction temperature of present invention may be comprised from 0° C. to 200° C. and preferably from 20° C. to 150° C.

The invented process may be carried out either in batch, semi-batch or in continuous mode. Furthermore, the process is not linked to a particular reactor type.

In one embodiment, ammonia could be optionally introduced into the reaction medium.

Should the disclosure of any patents, patent applications, and publications which are incorporated herein by reference conflict with the description of the present application to the extent that it may render a term unclear, the present description shall take precedence.

The following examples are included to illustrate embodiments of the invention. Needless to say, the invention is not limited to the described examples.

### EXPERIMENTAL PART

### Example 1

Raw materials furan-2,5-dicarbaldehyde dioxime, 2,5-bis(azidomethyl)furan and 2,5-bis(amino-methyl)furan used in these experiments were prepared from biomass derived platform chemical 5-hydromethylfurfural. Furan-2,5-dicarbaldehyde dioxime was prepared following the recipe of the prior arts (Hajj et. al. B. Soc. Chim. Fr., 1987, pp 855-860) and the others were prepared as follows.

To a 5 L round-bottomed flask equipped with a magnetic stir bar was added 2,5-bis(hydroxymethyl)furan (257 g, 2.0 mol) and a solution of diphenyl phosphoryl azide (1.2 kg, 4.4 mol) in toluene (3 L). Under stirring, DBU (732.7 g, 4.8 mol) was added drop-wise to the reaction mixture at 0° C., then warmed up to ambient temperature and stirred for 20 h. The reaction mixture was washed with water (2×4000 ml), HCl aqueous solution (5%, 4000 ml), and then brine (4000 ml). The organic layer was concentrated and 288 g (81% yield) of 2,5-bis(azidomethyl)furan as brown oil was obtained by distillation under vacuum (90° C./100 Pa).

Triethylamine (163.2 g, 1.6 mol) and hydrazine hydrate (564.5 g, 1.13 mol) were added to the solution of the diazide obtained above in methanol (6000 ml). Raney Ni (57.6 g) was added and the reaction mixture was stirred at 0° C. for 24 h. The catalyst was removed by filtration and the filtrate was concentrated under vacuum to give 170 g of 2,5-bis(aminomethyl)furan (pale yellow oil, 84% yield).

### Example 2

Into a 100 ml parr pressure reactor, 2,5-bis(azidomethyl)furan (1.50 g, 8.4 mmol) and 10% Pd/C (0.30 g) from Zhejiang Metallurgical Research Institute Co., Ltd were added and dissolved in methanol (50 ml). The mixture was stirred at 30° C. under 10 atms H₂ atmosphere for 18 h and was then filtered through celite after completion of the reaction. The filtrate was concentrated in vacuo to give 1.05 g of pale brown oil (90% isolated yield). ¹HNMR showed that 2,5-bis(aminomethyl) tetrahydrofuran was obtained in the ratio between cis and trans isomers of about 65:35.

### Example 3

Into a 30 ml autoclave, furan-2,5-dicarbaldehyde dioxime (153 mg, 1.0 mmol) and 5% Pd/C (47 mg) from Johnson Matthey were added and dissolved in methanol (5 ml). The mixture was stirred at 50° C. under 35 atms H₂ atmosphere for 18 h. The reaction mixture, after completion of the reaction, was analysed by GC. It was shown 2,5-bis(aminomethyl) tetrahydrofuran was obtained in 50% yield while the bicyclic compound 8-oxa-3-azabicyclo[3.2.1]octane appeared as byproduct in 5% yield.

### Example 4

Into a 30 ml autoclave, furan-2,5-dicarbaldehyde dioxime (312 mg, 2.0 mmol) and 5% Pd/Al₂O₃ (28 mg) from Johnson Matthey were added and dissolved in methanol (5 ml). The mixture was stirred at 100° C. under 45 atms H₂ atmosphere for 18 h. The reaction mixture, after completion of the reaction, was analysed by GC. It was shown 2,5-bis(aminomethyl) tetrahydrofuran was obtained in 50% yield while the bicyclic compound 8-oxa-3-azabicyclo[3.2.1]octane appeared as byproduct in 4% yield.

### Example 5

Into a 30 ml autoclave, 2,5-bis(aminomethyl)furan (134 mg, 1.06 mmol) and 5% Pd/C (50 mg) from Johnson Matthey were added and dissolved in methanol (5 ml). The mixture was stirred at 50° C. under 35 atms H₂ atmosphere for 18 h. The reaction mixture, after completion of the reaction, was analysed by GC and 2,5-bis(aminomethyl) tetrahydrofuran was obtained in 97% yield.

### Example 6

Into a 30 ml autoclave, 2,5-bis(aminomethyl)furan (127 mg, 1.01 mmol) and doped Raney Ni (120 mg) from Ningbo HanYi were added and dissolved in methanol (5 ml). The mixture was stirred at 60° C. under 20 atms H₂ atmosphere for 12 h. The reaction mixture, after completion of the reaction, was analysed by GC and 2,5-bis(aminomethyl) tetrahydrofuran was obtained in 89% yield.

### Comparative Example

Into a 30m1 autoclave, 2,5-bis(aminomethyl)furan (133 mg, 1.05 mmol) and doped Raney Ni (120 mg) from Ningbo HanYi were added and dissolved in p-xylene (5 ml). The mixture was stirred at 100° C. under 90 atms H₂ atmosphere for 12 h. The reaction mixture, after completion of the reaction, was analysed by GC and 2,5-bis(aminomethyl) tetrahydrofuran was obtained in 82% yield while the bicyclic compound 8-oxa-3-azabicyclo[3.2.1] octane appeared as byproduct in 6% yield.

This comparative example used same catalyst as Example 6 but was performed by at higher temperature and higher hydrogen gas pressure than Example 6. It illustrates that by using invented process, better yield of desired product could be obtained without relying on high temperature and gas pressure.

### Example 7

Into a 100 ml parr pressure reactor, furan-2,5-dicarbaldehyde dioxime (5.0 g, 32.4 mmol) and doped Raney Ni (1.0 g) from Ningbo HanYi were added and dissolved in methanol (40 ml). The mixture was stirred under 20 atms H₂ atmosphere at 50° C. for 24 h and then at 80° C. for another 24 h. The reaction mixture, after completion of the reaction, was analysed by ¹H NMR (biphenyl as internal standard) and GC-MS. It was shown 2,5-bis(aminomethyl) tetrahydrofuran was obtained in 75% yield while 2,5-bis(aminomethyl)furan intermediate appeared as a mixture with its oligomer in ca. 12% yield.

### Example 8

Into a 30 ml autoclave, 2,5-bis(aminomethyl)furan (240 mg, 1.9 mmol) and Raney Co (48 mg)(GRACE RANEY® 2724) were added and dissolved in methanol (5 ml). The mixture was stirred at 100° C. under 40 atms H₂ atmosphere for 12 h. The reaction mixture, after completion of the reaction, was analysed by GC and 2,5-bis(aminomethyl) tetrahydrofuran was obtained in 84% yield.

1. A process for preparing a tetrahydrofuran compound comprising at least two amine functional groups by reacting a furan compound comprising at least two nitrogen-containing functional groups with hydrogen in the presence of a hydrogenation catalyst, wherein the hydrogenation catalyst comprises:
at least one noble metal element in elemental form and/or at least one noble metal compound of at least one noble metal element, or
at least one metal element in elemental form and/or at least one metal compound of at least one metal element and a dopant, wherein the metal element is selected from the group consisting of (i) elements of group IA, IIA, IIIA, IVA, VA, VIA and VIIA of the Periodic Table, (ii) elements of groups IB, IIB, IIIB, IVB, VB, VIB, VIM and VIIIB of the Periodic Table, (iii) lanthanides, (iv) actinides, and (v) any combination thereof.

2. The process according to claim 1, wherein the noble metal element is selected from the group consisting of ruthenium, rhodium, palladium, silver, osmium, iridium, platinum and gold.

3. (canceled)

4. The process according to claim 1, wherein the hydrogenation catalyst is selected from the group consisting of Pd/C, Pt/C, Rh/C, Ru/C, Au/C, Pd/CeO₂, Pd/Al₂O₃, Pt/Al₂O₃, Rh/Al₂O₃, Ru/Al₂O₃ and Au/Al₂O₃.

5. The process according to claim 1, wherein the hydrogenation catalyst comprises one and only one metal element in elemental form and a dopant, wherein the only one metal element in elemental form is selected from the group consisting of elements of groups IB, IIB and VIIIB of the Periodic Table.

6. The process according to claim 1, wherein the hydrogenation catalyst comprises a mixture comprising at least two metal elements in elemental form and a dopant.

7. The process according to claim 1, wherein the hydrogenation catalyst comprises a metal alloy comprising at least two metal elements in elemental form and a dopant.

8. The process according to claim 6, wherein the two metal elements are selected from the group consisting of Pt, Pd, Rh, Ru, Au, Ag, Ni, Co, Fe, Cu, Sn, B, Al, Si, Sb, Bi and Ge.

9. The process according to claim 7, wherein the metal alloy is selected from the group consisting of Pt—Au, Pt—Pd, Pt—Sn, Pt—Bi, Pt—Fe, Rh—Ag, Rh—Au and Raney nickel alloys.

10. The process according to claim 9, wherein the hydrogenation catalyst is a doped Ni—Al Raney nickel alloy and the total aluminum content is comprised from 1% to 10% by weight with respect to the total weight of Ni—Al Raney nickel alloy.

11. The process according to claim 1, wherein the dopant is selected from the group consisting of elements of groups IVB, VB, VIB, VIIB and VIIIB of the Periodic Table.

12. The process according to 1, wherein the dopant is selected from the group consisting of Zn, Fe, Ti, Mo, V, Cr, Co, Mn, and combinations thereof.

13. The process according to claim 1, wherein hydrogenation catalyst is Ni—Al Raney nickel comprising metallic dopant elements iron and chromium.

14. The process according to claim 7, wherein the metal alloy is a Raney cobalt alloy.

15. The process according to claim 14, wherein the Raney cobalt alloy is a doped Co—Al Raney cobalt alloy and the total aluminum content is comprised from 1% to 10% by weight with respect to the total weight of the Co—Al Raney cobalt alloy.

16. The process according to claim 1, wherein the hydrogenation catalyst is a Co—Al Raney cobalt alloy comprising metallic dopant elements nickel and chromium.

17. The process according to claim 1, wherein the furan compound comprising at least two nitrogen-containing functional groups is selected from the group consisting of 2,5-bis(azidomethyl)furan, furan-2,5-dicarbaldehyde dioxime, 2,5-bis(hydrazonomethyl)furan, 2,5-bis(aminomethyl)furan, 3-((5-(aminomethyl)furan-2-yl)methoxy)propan-1-amine, 3,3′-((furan-2,5-diylbis(methylene))bis(oxy))bis(propan-1-amine), 3,3′-((furan-2,5-diylbis(methylene))bis(oxy))dipropanenitrile, ((oxybis(methylene))bis(furan-5,2-diyl))dimethanamine and 3,3′-((((oxybis(methylene))bis(furan-5,2-diyl))bis(methylene))bis(oxy))dipropanenitrile.

18. The process according to claim 1, wherein the tetrahydrofuran compound comprising at least two amine functional groups is selected from the group consisting of 2,5-bis(aminomethyl)tetrahydrofuran, 3-((5-(aminomethyl)tetrahydrofuran-2-yl)methoxy)propan-1-amine, 3,3′-(((tetrahydrofuran-2,5-diyl)bis(methylene))bis(oxy))bis(propan-1-amine), ((oxybis(methylene))bis(tetrahydrofuran-5,2-diyl))dimethanamine and 3,3′-((((oxybis(methylene))bis(tetrahydrofuran-5,2-diyl))bis(methylene))bis(oxy))bis(propan-1-amine).

19. The process according to claim 1, wherein the hydrogen has a gas pressure which is comprised from 5 to 80 bars.

20. The process according to claim 1, wherein the conversion of furan compound comprising at least two nitrogen-containing functional groups is comprised from 40% to 100%.

21. The process according to claim 1, wherein the selectivity of tetrahydrofuran compound comprising at least two amine functional groups is comprised from 60% to 100%.
